(19) United States
(12) Patent Application Publication
(10) Pub. No.: US 2004/0084384 A1
(43) Pub. Date: May 6, 2004

(54) Microbicidal separation systems

(76) Inventors: Peter Ottersbach, Windeck (DE); Friedrich Sosna, Dorsten (DE)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/469,534

(22) PCT Filed: Jan. 19, 2002
(86) PCT No.: PCT/EP02/00534

(30) Foreign Application Priority Data
Mar. 7, 2001 (DE) 101 10 885.0

(51) Int. Cl. C02F 1/68; A61K 31/74

(52) U.S. Cl. 210/764; 424/78.08; 210/501; 210/206

(57) ABSTRACT

The invention relates to microbicidal separation systems using antimicrobial polymers, and to their production and use.

The invention relates to the use of antimicrobial polymers for producing microbicidal separation systems.

It is highly undesirable for bacteria to become established or to spread on the surfaces of pipelines, containers, or packaging. Frequently, slime layers form and permit sharp rises in microbial populations, and these can lead to persistent impairment of the quality of water, drinks, or foods, and even to spoilage of the product, and harm to the health of consumers.

Bacteria must be kept away from all fields of life in which hygiene is important. This affects textiles for direct body contact, especially in the genital area, and those used for the care of the elderly or sick. Bacteria must also be kept away from surfaces of furniture and instruments in wards, especially in areas for intensive care and neonatal care, and in hospitals, especially in areas for medical intervention, and in isolation wards for critical cases of infection, and also in toilets.

There are also many industrial systems whose performance can be severely limited, or which can even be rendered entirely unusable, by the growth of microbes. Systems for separating materials, e.g. membranes or filters, are particularly severely impaired by the deposition and growth of microbes. For example, in sea water desalination the growth of marine algae in the systems shortens running times, often considerably. In other systems, e.g. deep-bed filtration, the filter cake can become blocked prematurely due to the growth of biofilms. Crossflow filtration attempts to counter this effect by using a specified flow perpendicular to the plane of filtration, but this has not proved adequate industrially for preventing the growth of biofilms.

A current method of treating equipment, or the surfaces of furniture or textiles, to resist bacteria either when this becomes necessary or else as a precautionary measure is to use chemicals or solutions of these, or else mixtures which are disinfectant and have fairly broad general antimicrobial action. Chemical agents of this type act nonspecifically and are themselves frequently toxic or irritant, or form degradation products which are hazardous to health. In addition, people frequently exhibit intolerance to these materials once they have become sensitized.

In the marine sector, the fouling of boats' hulls affects costs, since the growth of fouling organisms is attended by an increase in the boat's flow resistance, and thus by a marked increase in fuel consumption. Problems of this type have hitherto generally been countered by incorporating toxic heavy metals or other low-molecular-weight biocides into antifouling coatings, with the aim of mitigating the problems described. To this end, the damaging side effects of coatings of this type are accepted, but as society's environmental awareness rises, this state of affairs is increasingly problematic.

U.S. Pat. No. 4,532,269, for example, therefore discloses a terpolymer made from butyl methacrylate, tributyltin methacrylate, and tert-butylaminoethyl methacrylate. This copolymer is used as an antimicrobial paint for ships, and the hydrophilic tert-butylaminoethyl methacrylate promotes slow erosion of the polymer, thus releasing the highly toxic tributyltin methacrylate as active antimicrobial ingredient.

In these applications, the copolymer prepared with amino methacrylates is merely a matrix or carrier for added microbicidal active ingredients which can diffuse or migrate out of the carrier material. At some stage polymers of this type lose their activity, once the necessary minimum inhibitor concentration (MIC) is no longer-achieved at the surface.

It is also known from the European Patent Application 0 862 858 that copolymers of tert-butylaminoethyl methacrylate, which is a methacrylate with a secondary amino function, has inherent microbicidal properties. This terpolymer has what are known as contact-microbicidal properties without addition of any microbicidal active ingredient. A wide variety of contact-microbicidal polymers is known from the following Patent Applications: DE 100 24 270, DE 100 22 406, PCT/EP00/06501, DE 100 14 726, DE 100 08 177, PCT/EP00/06812, PCT/EP00/06487, PCT/EP00/06506, PCT/EP00/02813, PCT/EP00/02819, PCT/EP00/02818, PCT/EP00/02780, PCT/EP00/02781, PCT/EP00/02783, PCT/EP00/02782, PCT/EP00/02799, PCT/EP00/02798, PCT/EP00/00545, PCT/EP00/00544.

These polymers comprise no low-molecular weight constituents. The antimicrobial properties are attributable to contact between bacteria and the surface.

Polymers which have contact-microbicidal action without addition of low-molecular-weight biocides are hereinafter termed antimicrobial polymers.

Systems developed in the future will also have to be based on novel compositions with improved effectiveness if undesirable resistance phenomena in microbes are to be avoided, particularly bearing in mind the microbial resistance known from antibiotics research. Application-related technical issues also play an important part here, since the antimicrobial polymers are often processed together with other plastics in order to strengthen their resistance to microbiological attack or, in the ideal case render them completely inert.

The use of biocides is intrinsically impossible in many material separation systems, since when they are operating, e.g. filtering foods or drinks such as beer, it is not permissible to add any substance which could result in contamination of the product and, in extreme cases, even lead to poisoning. It has hitherto been impossible in such cases to avoid shutdown of the plants with all of the associated high costs, since the biofilms have to be removed mechanically or, where appropriate, by toxicologically non-hazardous chemical means. Even in purely industrial systems which do not come into contact with food or drink, it is often impossible to use low-molecular-weight biocides, since these substances generally require complicated disposal after use.

It is an object of the present invention, therefore, to provide separation systems which themselves can substantially prevent microbial infestation. These separation systems should moreover not release any toxic substances, and should therefore ultimately be compatible with food and drink.

It has been found that separation systems, i.e. membranes, sieves or filters, produced from or with antimicrobial polymers can combine good separation performance with excellent antimicrobial action.

The present invention therefore provides separation systems comprising antimicrobial polymers.

Separation systems of the invention may be membranes, filters, sieves, or oxygenator modules, with pore widths extending from the nanometer to the millimeter range (from below 1 nm to 2 mm).

The separation systems may be composed entirely of the antimicrobial polymers, of a mixture (blend) of the antimicrobial polymers with at least one other polymer, or of a prefabricated separation system with a coating made from antimicrobial polymers or, respectively, the abovementioned polymer blend. Prefabricated polymer-based separation systems may, for example, be impregnated in regions near to the surface by a solution of the antimicrobial polymer in a suitable solvent which can also be a suitable swelling agent for the polymer base.

The prefabricated, i.e. non-antimicrobial, separation systems may in turn be composed of polymers, of ceramics, or of metals.

In all cases use is made of at least one antimicrobial polymer. It can be advantageous again here to use a polymer blend made from various antimicrobial polymers.

Processes which may be used jointly for antimicrobial and non-antimicrobial, conventional polymers are in principle any of the processes used for plastics, e.g. the production of a joint compound, processing by means of coextrusion, or else incorporation into coating systems or paint systems.

The preferred method of rendering polymer membranes antimicrobial is the addition of antimicrobial polymers in the process to produce the membrane. The method of achieving this is generally that antimicrobial polymer is added to the melt or the solution comprising the plastic and from which the polymer membrane is to be manufactured, and is homogenized in this plastic. The mixture is then placed in a mold, dried, and stretched at an elevated temperature in order to obtain the desired pore sizes and distributions in the membrane. As an alternative to this, the pores may also be obtained in another way, in particular by using gamma radiation or other high-energy electromagnetic radiation.

This procedure gives an antimicrobial membrane which combines together, in a manner which is close to ideal, the required separation performance for the tasks to be undertaken and the biochemical inhibitory action with respect to microbe growth. Since the antimicrobial polymer has been fixed within the matrix of the membranes and therefore no low-molecular-weight constituents are released into the flow system, membranes of this type may also be used in sensitive areas, e.g. the processing of food or drink, without giving rise to concerns that biocides may pass into the product and cause a toxic hazard. Shutdown times for plants can thus be further reduced and plants can be designed without a need to consider crossflow, as used in the crossflow filtration mentioned.

The antimicrobial polymers are preferably prepared using nitrogen- or phosphorus-functional monomers, and these polymers are in particular prepared from at least one of the following monomers: 2-tert-butylaminoethyl methacrylate, 2-diethylaminoethyl methacrylate, 2-diethylaminomethyl methacrylate, 2-tert-butylaminoethyl acrylate, 3-dimethylaminopropyl acrylate, 2-diethylaminoethyl acrylate, 2-dimethylaminoethyl acrylate, dimethylaminopropylmethacrylamide, diethylaminopropylmethacrylamide, N-3-dimethylaminopropylacrylamide, 2-methacryloyloxyethyltrimethylammonium methosulfate, 2-diethylaminoethyl methacrylate, 2-methacryloyloxyethyltrimethylammonium chloride, 3-methacryloylaminopropyltrimethylammonium chloride, 2-methacryloyloxyethyltrimethylammonium chloride, 2-acryloyloxyethyl-4-benzoyidimethylammonium bromide, 2-methacryloyloxyethyl-4-benzoyidimethylammonium bromide, 2-acrylamido-2-methyl-1-propanesulfonic acid, 2-diethylaminoethyl vinyl ether, 3-aminopropyl vinyl ether.

The proportion of the antimicrobial polymers in the separation systems may be from 0.01 to 25% by weight, preferably from 0.1 to 10% by weight, particularly preferably from 0.1 to 5% by weight.

Other polymers used, i.e. non-antimicrobial polymers, may in principle be any of the macromolecules usually used for producing polymer membranes, in particular polyethylene, polypropylene, polymethacrylates, polysulfones, polyacrylonitrile, cellulose, cellulose acetate, or other cellulose derivatives. Like all of the other hydrophilic polymer membranes, the cellulose derivatives have the advantage that no microdomain formation is to be expected with the antimicrobial polymers, which are often also hydrophilic, and this makes it easier to obtain uniform surface availability of the antimicrobial polymers.

### Use of the Modified Polymer Substrates

The present invention also provides the use of the microbicidal membranes produced according to the invention as a part of filter systems or of filter modules.

The separation systems of the invention may be used for filtering beer, wine, fruit juices, milk, or drinking water, or else as a liquid/gaseous separation system (oxygenator module).

The examples below are given for further description of the present invention and give further illustration of the invention but are not intended to limit its scope as set out in the claims.

### EXAMPLE 1

50 ml of dimethylaminopropylmethacrylamide (Aldrich) and 250 ml of ethanol are charged to a three-necked flask and heated to 65° C. under a stream of argon. 0.5 g of azobisisobutyronitrile dissolved in 20 ml of ethanol are then slowly added dropwise, with stirring. The mixture is heated to 70° C. and stirred at this temperature for 6 hours. Once this time has expired, the solvent is distilled off from the reaction mixture, and this is followed by drying for 24 hours at 50° C. in vacuo. The product is then dissolved in 200 ml of acetone, then the solvent is distilled off from the reacton mixture, and this is followed by drying for 24 hours at 50° C. in vacuo. The reaction product is then finely ground using a pestle and mortar.

### EXAMPLE 1a

50 g of polypropylene are heated to 180° C. and intimately mixed with 3 g of the product from Example 1. The polymer mixture is processed, while still hot, through a laboratory calender, to give a plastic film of about 100 micrometers thickness. The cooled film is reheated to 170° C. and subjected to mechanical stretching, producing the pores in the membrane. The membrane is then allowed to cool to room temperature.

### EXAMPLE 1b

A piece of the plastic membrane from Example 1a, of dimensions 3×3 cm, is placed on the base of a glass beaker which contains 10 ml of a test microbial suspension of Pseudomonas aeruginosa, and the resultant system is then shaken for 4 hours. 1 ml of the test microbial suspension is then removed. After expiry of this period, the number of microbes per ml has fallen from 10⁷ to 10³.

### EXAMPLE 2

50 ml of tert-butylaminoethyl methacrylate (Aldrich) and 250 ml of ethanol are charged to a three-necked flask and heated to 65° C. under a stream of argon. 0.5 g of azobisisobutyronitrile dissolved in 20 ml of ethanol are then slowly added dropwise, with stirring. The mixture is heated to 70° C. and stirred at this temperature for 6 hours. Once this time has expired, the solvent is distilled off from the reaction mixture, and this is followed by drying the product for 24 hours at 50° C. in vacuo. The product is then dissolved in 200 ml of acetone, then the solvent is distilled off from the reacton mixture, and this is followed by drying for 24 hours at 50° C. in vacuo.

### EXAMPLE 2a

50 g of polypropylene are heated to 180° C. and intimately mixed with 3 g of the product from Example 2. The polymer mixture is processed, while still hot, through a laboratory calender, to give a plastic film of about 100 micrometers thickness. The cooled film is reheated to 170° C. and subjected to mechanical stretching, producing the pores in the membrane. The membrane is then allowed to cool to room temperature.

### EXAMPLE 2b

A piece of the plastic membrane from Example 2a, of dimensions 3×3 cm, is placed on the base of a glass beaker which contains 10 ml of a test microbial suspension of Pseudomonas aeruginosa, and the resultant system is then shaken for 4 hours. 1 ml of the test microbial suspension is then removed. After expiry of this time, no remaining Pseudomonas aeruginosa microbes are detectable.

What is claimed is:

1. An antimicrobial separation system comprising antimicrobial polymers.

2. The antimicrobial separation system as claimed in claim 1, wherein
the separation system is composed of a polymer blend made from at least one antimicrobial polymer and from at least one other polymer.

3. The antimicrobial separation system as claimed in claim 1, wherein
the separation system is composed of a non-antimicrobial separation system coated with at least one antimicrobial polymer.

4. The antimicrobial separation system as claimed in claim 3, wherein
the non-antimicrobial separation system is composed of ceramics, polymers, or metal.

5. The antimicrobial separation system as claimed in any of claims 1 to 4, wherein
the separation system is a membrane, a filter, or a sieve.

6. The antimicrobial separation system as claimed in any of claims 1 to 5, wherein
the separation system comprises from 0.01 to 25% by weight of the antimicrobial polymer.

7. The antimicrobial separation system as claimed in any of claims 1 to 6, wherein
the antimicrobial polymers have been prepared from at least one of the following monomers:
2-tert-butylaminoethyl methacrylate, 2-diethylaminoethyl methacrylate, 2-diethylaminomethyl methacrylate, 2-tert-butylaminoethyl acrylate, 3-dimethylaminopropyl acrylate, 2-diethylaminoethyl acrylate, 2-dimethylaminoethyl acrylate, dimethylaminopropylmethacrylamide, diethylaminopropylmethacrylamide, N-3-dimethylaminopropylacrylamide, 2-methacryloyloxyethyltrimethylammonium methosulfate, 2-diethylaminoethyl methacrylate, 2-methacryloyloxyethyltrimethylammonium chloride, 3-methacryloylaminopropyltrimethylammonium chloride, 2-methacryloyloxyethyltrimethylammonium chloride, 2-acryloyloxyethyl-4-benzoyldimethylammonium bromide, 2-methacryloyloxyethyl-4-benzoyidimethylammonium bromide, allyltriphenylphosphonium bromide, allyltriphenylphosphonium chloride, 2-acrylamido-2-methyl-1-propanesulfonic acid, 2-diethylaminoethyl vinyl ether, 3-aminopropyl vinyl ether.

8. The antimicrobial separation system as claimed in any of claims 1 to 7, wherein
a separation system also comprises, besides at least one antimicrobial polymer, polyethylene, polypropylene, polymethacrylates, polysulfones, polyacrylonitrile, cellulose, cellulose acetate, or cellulose derivatives.

9. The use of microbicidal polymer membranes as claimed in any of claims 1 to 8, as a part of filter systems or of filter modules.

10. The use of the separation system as claimed in any of claims 1 to 8, for filtering beer or wine.

11. The use of the separation system as claimed in any of claims 1 to 8, as a liquid/gaseous separation system (oxygenator module).

12. The use of the separation system as claimed in any of claims 1 to 8, for filtering fruit juices, milk, or drinking water.
